(19) United States
(12) Patent Application Publication
(10) Pub. No.: US 2025/0269110 A1
(43) Pub. Date: Aug. 28, 2025

(54) AN INFUSION SUBASSEMBLY AND AN INFUSION ASSEMBLY

(71) Applicant: SHL Medical AG, Zug (CH)

(72) Inventors: Hsueh-Yi Chen, Taoyuan City (TW); Chun-Yi Lee, Taoyuan City (TW); Shih-Hsun Tu, Taoyuan City (TW); Tai-Fu Wang, Taoyuan City (TW)

(21) Appl. No.: 18/703,140

(22) PCT Filed: Oct. 11, 2022
(86) PCT No.: PCT/EP2022/078201

§ 371 Date: Apr. 19, 2024

(30) Foreign Application Priority Data
Oct. 25, 2021 (EP) 21204513.2

(51) Int. Cl. A61M 5/142

(52) U.S. Cl. CPC A61M 5/14232; A61M 5/14244; A61M 2005/14256

(57) ABSTRACT

The present disclosure provides an infusion subassembly, comprising a pump, and a user wearable garment including a protruding support portion supporting the pump to the user wearable garment. The user wearable garment further includes a container supporting portion, the container supporting portion being configured to support a medicament container to the user wearable garment. The container supporting portion comprises an end surface, the end surface comprising an end portion and an opening. The pump is connectable to the end portion of the end surface, and wherein the pump is configured to force medicament from the medicament container to be dispensed through the opening of the end surface.

### CROSS-REFERENCE TO RELATED APPLICATIONS

The present application is a U.S. National Phase Application pursuant to 35 U.S.C. § 371 of International Application No. PCT/EP2022/078201 filed Oct. 11, 2022, which claims priority to European Patent Application No. 21204513.2 filed Oct. 25, 2021. The entire disclosure contents of these applications are herewith incorporated by reference into the present application.

### TECHNICAL FIELD

The present disclosure generally relates to an infusion subassembly and an infusion assembly for delivery of medicament to a medicament recipient.

### BACKGROUND

Medicament is conventionally expelled from a medicament delivery device to a so-called dose delivery site, e.g. through the skin of a medicament recipient in need of the medicament. The medicament delivery device is often arranged as a syringe for administrating the medicament. The medicament is expelled from the syringe by manually pushing a plunger for forcing the medicament out through a needle and into the medicament recipient.

For some types of medicament, such as e.g. oncology medicament, the medicament is injected by a healthcare professional. The healthcare professional is thus holding the syringe and manually expels the medicament into the medicament recipient.

However, and with reference to the exemplified oncology drugs, the medicament is expelled from the medicament delivery device into the medicament recipient during a relatively long period of time. This duration of injection is associated with quite some strains to the healthcare professional as it requires manual efforts not seldom in an uncomfortable working position. Time consuming medicament delivery to a medicament recipient is thus in need of further improvements to streamline and facilitate the work performed by the healthcare professional.

### SUMMARY

The invention is defined by the appended claims, to which reference should now be made.

In the present disclosure, when the term “distal direction” is used, this refers to the direction pointing away from the dose delivery site during use of the medicament delivery device. When the term “distal part/end” is used, this refers to the part/end of the delivery device, or the parts/ends of the members thereof, which under use of the medicament delivery device is/are located furthest away from the dose delivery site. Correspondingly, when the term “proximal direction” is used, this refers to the direction pointing towards the dose delivery site during use of the medicament delivery device. When the term “proximal part/end” is used, this refers to the part/end of the delivery device, or the parts/ends of the members thereof, which under use of the medicament delivery device is/are located closest to the dose delivery site.

Further, the term “longitudinal”, “longitudinally”, “axially” or “axial” refer to a direction extending from the proximal end to the distal end, typically along the device or components thereof in the direction of the longest extension of the device and/or component.

Similarly, the terms “transverse”, “transversal” and “transversally” refer to a direction generally perpendicular to the longitudinal direction.

Further, the terms “circumference”, “circumferential”, or “circumferentially” refer to a circumference or a circumferential direction relative to an axis, typically a central axis extending in the direction of the longest extension of the device and/or component. Similarly, “radial” or “radially” refer to a direction extending radially relative to the axis, and “rotation”, “rotational” and “rotationally” refer to rotation relative to the axis.

According to a first aspect of the present disclosure there is provided an infusion subassembly, comprising a pump, and a user wearable garment, comprising a protruding support portion supporting the pump to the user wearable garment, and a container supporting portion, the container supporting portion being configured to support a medicament container to the user wearable garment, wherein the container supporting portion comprises an end surface, the end surface comprising an end portion and an opening, wherein the pump is connectable to the end portion of the end surface, and wherein the pump is configured to force medicament from the medicament container to be dispensed through the opening of the end surface.

The user wearable garment should be construed as a piece of garment that should preferably be worn by a healthcare professional that is performing the injection of medicament to a medicament recipient. Detailed example embodiments of the user wearable.

The present disclosure is based on the insight that by providing a pump and a container supporting portion, to which a medicament container is insertable, to the user wearable garment, the healthcare professionals do no longer need to hold the medicament container in his/her hand when injecting the medicament. The pump also enables for expulsion of medicament without the need of e.g. manually pushing a plunger. The infusion subassembly thus advantageously enables for an ergonomic and efficient delivery of medicament to the medicament recipient.

According to an exemplified embodiment, the pump may be connectable to the end portion of the container supporting portion by an elongated tube between the pump and the end portion. Hereby, pump can be arranged at a distance from the container supporting portion. The pump and the container supporting portion are hereby arranged at spaced apart positions on the user wearable garment.

According to an exemplified embodiment, the protruding support portion and the container supporting portion may be integrally formed with the user wearable garment. Hereby, well defined portions for arranging the pump as well as the medicament container is provided on the user wearable garment. Also, and in comparison to an externally and non-integrated protruding support portion and container support portion, less components are provided and e.g. the healthcare professionals do not need to perform an assembly process before usage.

According to an exemplified embodiment, the infusion subassembly may further comprise a control unit connected to the pump, wherein the control unit comprises control circuitry configured to transmit a control signal to the pump, the control signal representing instructions which, when executed by the pump, cause activation of the pump to force transfer of medicament from the medicament container out through the opening of the end surface. The control unit thus enables for even less interaction from the user.

According to an exemplified embodiment, the control unit may be embedded in the user wearable garment. By embedding the control unit in the user wearable garment, a less bulky infusion subassembly is provided.

According to an exemplified embodiment, the infusion subassembly may further comprise an actuator arranged on the user wearable garment, the actuator being connected to the control unit, and wherein the control signal to the pump is transmitted upon actuation of the actuator. Hereby, the user, e.g. the healthcare professional can simply push the actuator for initiating the medicament delivery process.

According to an exemplified embodiment, the infusion subassembly may further comprise at least one battery connectable to the pump, wherein the battery is supported by the protruding support portion. The protruding support portion thus serves the dual function of supporting the pump as well as the batteries. The replacement of the batteries can hereby be simplified since the connection to the protruding support portion provides a simple access. The protruding portion preferably comprises circuitry for electrically connecting the at least one battery to the pump.

According to an exemplified embodiment, the pump may comprise a pump inlet connectable to the end portion of the container supporting portion. Preferably, the elongated tube is connectable between the pump inlet and the container supporting portion.

According to an exemplified embodiment, the pump may comprise at least one engagement roller, the at least one engagement roller being configured to, during rotation around a rotation axis of the at least one engagement roller, squeeze a portion of the elongated tube. Preferably, the portion of the elongated tube is under compression by the at least one engagement roller. The portion of the elongated tube exposed to the compression is preferably “closed”, i.e. inner walls of the portion under compression are pressed against each other. The medicament is hereby forced away in a direction from the container support portion to e.g. a needle insertion arrangement by the rotation of the at least one engagement roller.

According to an exemplified embodiment, the pump may comprise a first housing portion configured to house the portion of the elongated tube, wherein the at least one engagement roller is configured to squeeze the portion of the elongated tube against a surface of the first housing portion. The portion of the elongated tube is hereby closed between the at least one engagement roller and the surface of the first housing portion.

According to an exemplified embodiment, the at least one engagement roller may be formed by a first engagement roller and a second engagement roller, the first and second engagement rollers being configured to, during rotation of the first engagement roller around a first rotation axis and rotation of the second engagement roller around a second rotation axis, squeeze the portion of the elongated tube between the first and second engagement rollers. Hereby, the two engagement rollers are in conjunction with each other closing the portion of the elongated tube, whereby the rotation of the two engagement rollers forces transportation of the medicament.

According to an exemplified embodiment, the pump may further comprise an electric motor connected to the at least one engagement roller.

According to an exemplified embodiment, the electric motor may comprise a motor shaft connected to the at least one engagement roller for rotating the at least one engagement roller.

According to an exemplified embodiment, the pump may comprise a second housing portion, the electric motor being arranged in the second housing portion.

According to an exemplified embodiment, the protruding support portion may comprise a pump supporting surface, wherein the pump is arranged in an opening of the pump supporting surface, and wherein the first and second housing portions are arranged on opposite sides of the pump supporting surface.

Hereby, the pump and the electric motor are arranged on opposite sides of the pump supporting surface, which simplifies the accessibility for each of the pump and the electric motor. The above described batteries may hereby be electrically connected to the electric motor supply of electric power thereto.

According to an exemplified embodiment, the at least one engagement roller may comprise a rotatable friction surface, the rotatable friction surface being configured to squeeze the portion of the elongated tube against the surface of the first housing portion upon rotation of the at least one engagement roller. The friction surface will increase the functional operation of the at least one engagement roller when squeezing the portion of the elongated tube, ensuring that the portion of the elongated tube is prevented from sliding when engaging with the at least one engagement roller.

According to an exemplified embodiment, the pump may be a peristaltic pump.

According to an exemplified embodiment, the container supporting portion may be an elongated container supporting portion, and wherein the opening is a first opening, and the end portion is a first end, the elongated container supporting portion further comprising a second opening at a second end, the elongated container supporting portion extending between the first end and the second end, wherein the medicament container is insertable to the elongated container supporting portion from the second opening. Hereby, the container supporting portion is arranged in the form of a pocket for properly securing the medicament container to the user wearable garment.

According to an exemplified embodiment, the elongated container supporting portion may extend, during use of the infusion subassembly, between the first end and the second end in an at least partially vertical direction relative to ground. Hereby, the medicament container is, due to gravity, sufficiently prevented from falling out from the container supporting portion. Also, the medicament in the medicament container will, during use of the infusion subassembly, be accessible from the first opening.

According to an exemplified embodiment, the user wearable garment may comprise a base portion, and two arm portions, wherein each arm portion extends from the base portion. The arm portions may preferably be arranged in a shape such that the two arm portions are provided around the neck of the user wearing the user wearable garment. The base portion is thus preferably arranged on the chest of the user wearing the user wearable garment. Accordingly, and according to an exemplified embodiment, the first and second arm portions may, together with the base portion, be configured to form a closed loop.

According to an exemplified embodiment, the base portion and the arm portions may be integrally formed with each other. The base portion and the two arm portions are hereby formed in one piece, reducing the need of also assembling the user wearable garment before usage.

According to an exemplified embodiment, the protruding support portion and the container supporting portion may be arranged on the base portion.

According to an exemplified embodiment, the protruding support portion and the container supporting portion may be arranged on a lower end portion of the base portion and the two arm portions are arranged on an upper end portion of the base portion, the upper and lower end portions being arranged on opposite vertical ends of the base portion, wherein the two arms extend in a direction away from the lower end portion.

According to an exemplified embodiment, a first one of the two arm portions may comprise a first connector element, and wherein a second one of the two arm portions comprises a second connector element, the first and second connector elements being connectable to each other to form the closed loop.

According to an exemplified embodiment, the first connector element may be arranged on the first arm portion at a first position away from the base portion, and wherein the second connector element is arranged on the second arm portion at a second position away from the base portion.

The first and second connector elements are hereby arranged on a free end of the respective arm portions. The user of the wearable garment can hereby connect the first and second connector elements to each other at the back of his/her neck. Accordingly, and according to an exemplified embodiment, the first connector element may be arranged at an outer end portion of the first arm portion, and the second connector element is arranged at an outer end portion of the second arm portion.

According to an exemplified embodiment, the first connector element may be a male connector element and the second connector element is a female connector element, the male connector element is arranged to mate with the female connector element.

According to an exemplified embodiment, the pump may be directly connectable to the end portion of the container supporting portion.

According to an exemplified embodiment, the pump may be an air compressor pump, the end surface of the container supporting portion comprising an additional opening arranged adjacent the opening, wherein an air outlet portion of the air compressor is connectable to the additional opening for supplying pressurized air to the medicament container. An alternative pump is here provided which in some implementations may be more suitable to be used compared to a pump using e.g. at least one engagement roller.

According to a second aspect, there is provided an infusion assembly, comprising an infusion subassembly according to any one of the embodiments described above in relation to the first aspect, a medicament container supported by the container supporting portion and connected to the pump, a needle insertor arrangement comprising an inlet, and an elongated tube extending between a first end and a second end, wherein the first end is attached to the end portion of the container supporting portion, and the second end is attached to the inlet of the needle insertor arrangement.

An advantage of the second aspect is that the above described infusion subassembly can be used in conjunction with the needle insertor arrangement. The needle insertor arrangement can be arranged in abutment with the skin of the medicament recipient. Preferably, the needle insertor arrangement can maintain stationary onto the skin of the medicament recipient when administrating the medicament, thereby reducing the active involvement of the healthcare professional holding any parts of the infusion assembly to the skin.

According to an exemplified embodiment, an aspiration needle may be attached to the first end of the elongated tube, the aspiration needle being penetrated through a lid of the medicament container.

According to an exemplified embodiment, the needle insertor arrangement may comprise an injection site abutment surface configured to be arranged in abutment with a medicament recipient subject to medicament treatment. The injection site abutment surface may preferably be provided with an adhesive, thereby keeping the needle insertor arrangement in position on the skin of the medicament recipient during administration of the medicament.

Further effects and features of the second aspect are largely analogous to those described above in relation to the first aspect.

According to a third aspect, there is provided a method of controlling medicament delivery to a medicament recipient using an infusion assembly according to any one of the embodiments described above in relation to the second aspect, wherein the method comprises the steps of positioning an injection site abutment surface of the needle insertor arrangement in abutment with a skin surface of a medicament recipient subject to medicament treatment, attaching the first end portion of the elongated tube to the medicament container, attaching the second end portion of the elongated tube to the inlet portion of the needle insertor arrangement; controlling the needle insertor arrangement to force a penetration needle of the needle insertor arrangement to protrude from the injection site abutment surface; and controlling the pump to transfer medicament from the medicament container to the penetration needle via the inlet portion of the needle insertor arrangement.

According to an exemplified embodiment, the step of attaching the second end portion of the elongated tube to the inlet portion of the needle insertor arrangement may be preceded by the steps of verifying a flow of medicament from the medicament container through the elongated tube by activating the pump; and de-activating the pump when medicament flows out from the second end portion of the elongated tube. Hereby, the infusion assembly can be tested to verify that medicament is delivered sufficiently before actual usage.

Further effects and features of the third aspect are largely analogous to those described above in relation to the first and second aspects.

Generally, all terms used in the claims are to be interpreted according to their ordinary meaning in the technical field, unless explicitly defined otherwise herein. All references to “a/an/the member, apparatus, component, means, etc.” are to be interpreted openly as referring to at least one instance of the member, apparatus, component, means, etc., unless explicitly stated otherwise.

### BRIEF DESCRIPTION OF THE DRAWINGS

The specific embodiments of the inventive concept will now be described, by way of example, with reference to the accompanying drawings, in which:

FIG. 1A is a schematic illustration of an infusion assembly, including an exemplified infusion subassembly, according to an exemplified embodiment during usage thereof,

FIG. 1B is schematic illustration of the infusion assembly, where the infusion subassembly is arranged on the medicament recipient according to an exemplified embodiment,

FIG. 2 is a perspective view of the infusion assembly and the infusion subassembly according to an exemplified embodiment,

FIG. 3 is an exploded view and an assembled view of the pump of the infusion assembly according to an exemplified embodiment,

FIG. 4 is a schematic illustration the interaction between a medicament container and an elongated tube of the infusion assembly according to an exemplified embodiment,

FIG. 5 is an exploded view of an example needle insertor arrangement according to an exemplified embodiment,

FIG. 6 is an exploded view of the needle insert arrangement according an exemplified embodiment,

FIG. 7 is an exploded view of a cannula assembly and a medicament path assembly according to an exemplified embodiment,

FIG. 8 is an exploded view of a needle assembly, the cannula assembly, and medicament path assembly as well as a perspective view of said components assembled according to an exemplified embodiment,

FIG. 9 is an exploded view of a torsion spring, a rotator, and a sleeve as well as a perspective view of said components assembled according to an exemplified embodiment,

FIG. 10 is an exploded view of a base, a piercer, the cannula assembly, a driver, the needle assembly, and the medicament path assembly according to an exemplified embodiment,

FIG. 11 is a perspective view of the base, the piercer, the driver, the needle assembly, the cannula assembly, and the medicament path assembly assembled according to an exemplified embodiment,

FIG. 12 is a perspective view of a movable stop to be assembled with the base according to an exemplified embodiment,

FIG. 13 is a perspective view of the torsion spring, the rotator, and the sleeve to be assembled with the base according to an exemplified embodiment,

FIG. 14 is a cross-section view of the needle insertor arrangement before the rotator is released according to an exemplified embodiment,

FIG. 15 is a cross-section view of the needle insertor arrangement when the rotator is rotated by associated torsion spring according to an exemplified embodiment,

FIG. 16 is a cross-section view of the needle insertor arrangement when the rotator retracts the needle portion back inside the case and is prevented from further rotating according to an exemplified embodiment,

FIG. 17 is an exploded view of an example needle insertor arrangement according to another exemplified embodiment of the present disclosure,

FIG. 18 is a perspective view of the needle insertor arrangement in FIG. 17 according to an exemplified embodiment,

FIG. 19 is a further perspective view of the needle insertor arrangement of FIGS. 17 and 18 according to an exemplified embodiment, and

FIG. 20 is a flow chart of a method of controlling medicament delivery to a medicament recipient according to an exemplified embodiment.

### DETAILED DESCRIPTION

The inventive concept will now be described more fully hereinafter with reference to the accompanying drawings, in which exemplifying embodiments are shown. The inventive concept may, however, be embodied in many different forms and should not be construed as limited to the embodiments set forth herein; rather, these embodiments are provided by way of example so that this disclosure will be thorough and complete, and will fully convey the scope of the inventive concept to those skilled in the art. Like numbers refer to like members throughout the description.

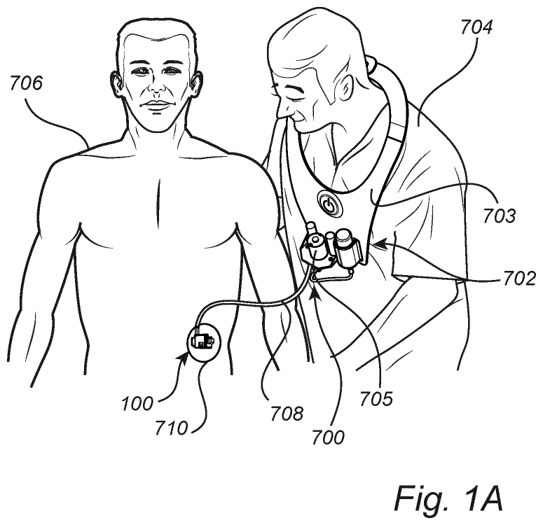

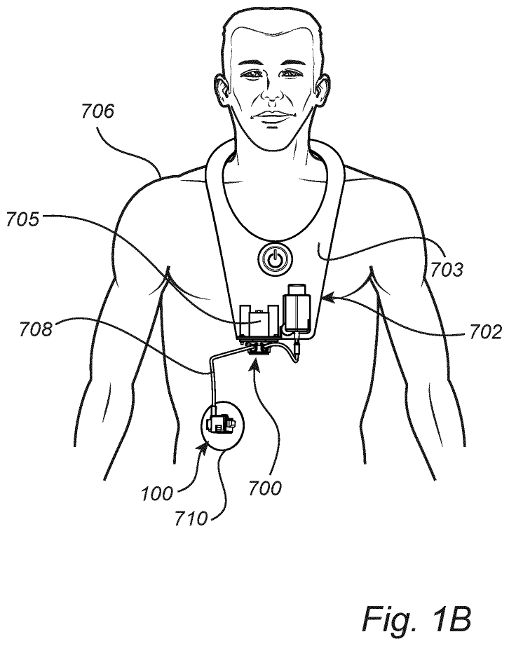

Reference is made to FIG. 1A which is a schematic illustration of an infusion assembly 700 according to an exemplified embodiment. The infusion assembly 700 comprises an infusion subassembly 702. The infusion subassembly 702 comprises a pump 705 and a user wearable garment 703 supporting the pump 705. The user wearable garment 703 is exemplified as arranged on a healthcare professional 704 administrating a delivery of medicament to a medicament recipient 706. In further detail, the infusion subassembly 702 is connected to a needle insertor arrangement 100 by means of an elongated tube 708. The needle insertor arrangement 100 is in turn arranged in abutment with a dose delivery site 710 of the medicament recipient 706, i.e. the skin of the patient receiving the medicament.

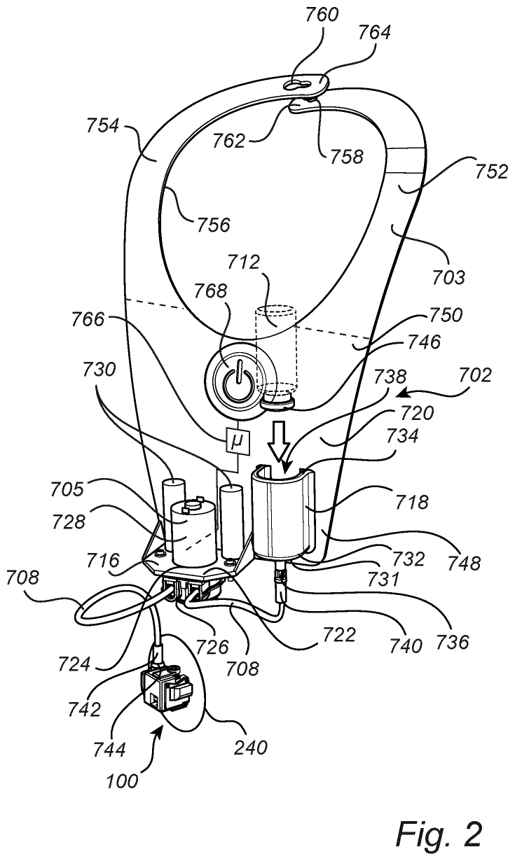

With reference to FIG. 1B which illustrates an exemplified embodiment in which the infusion subassembly 702 is arranged on the medicament recipient 706 instead of the healthcare professional 704 as described above in relation to FIG. 1A. Thus, the medicament recipient 706, i.e. the patient, can advantageously also wear the user wearable garment and administrate the delivery of medicament to himself/herself. The infusion assembly 700 and the infusion subassembly 702, as well as their associated components, will be described in further detail below with reference to FIGS. 2-4.

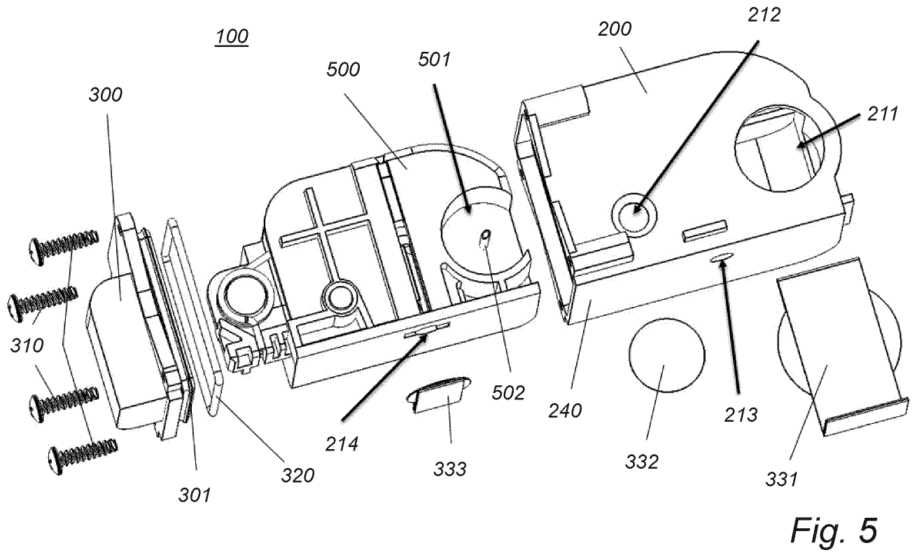

Reference is now made to FIG. 2, which is a perspective view of the infusion assembly 700 and the infusion subassembly 702 according to an exemplified embodiment. As described above, the infusion assembly 700 comprises the infusion subassembly 702, the elongate tube 708 and the needle insertor arrangement 100. The needle insertor arrangement 100, which will be described in further detail below with reference to FIGS. 5-19, comprises an injection site abutment surface 240. The injection site abutment surface 240 is configured to be arranged in abutment with the medicament recipient 706. In detail, the injection site abutment surface 240 is configured to be arranged in abutment with the dose delivery site (710 in FIG. 1A) of the medicament recipient 706. According to a non-limiting example, the injection site abutment surface 240 comprises an adhesive for improving the attachment of the needle insertor arrangement 100 to the skin of the medicament recipient 706. The infusion assembly 700 further comprises a medicament container 712 configured to contain medicament for delivery to the medicament recipient (706 in FIG. 1A).

As described above, the infusion subassembly 702 comprises a user wearable garment 703. The user wearable garment 703 comprises a protruding support portion 716 and a container supporting portion 718. The protruding support portion 716 is arranged to support the pump 705 to the user wearable garment 703, while the container supporting portion 718 is configured to support the medicament container 712 to the user wearable garment 703. Preferably, the protruding support portion 716 and the container supporting portion 718 are integrally formed with the user wearable garment 703, and spaced apart from each other. A base portion 720 of the user wearable garment 703 is thus formed in one piece with the protruding support portion 716 and the container supporting portion 718.

Furthermore, the protruding support portion 716 comprises a pump supporting surface 722. The pump 705 is arranged in an opening 724 of the pump supporting surface 722. The pump 705 further comprises a first housing portion 726 and a second housing portion 728, where the first 726 and second 728 housing portions are arranged on opposite sides of the pump supporting surface 722. In particular, and during use of the infusion assembly 700, the first housing portion 726 is arranged vertically below the pump supporting surface 722 relative to ground, while the second housing portion 728 is arranged vertically above the pump supporting surface 722 relative to ground.

Moreover, the infusion subassembly 702 also comprises at least one battery 730. In the exemplified example of FIG. 2, the infusion subassembly 702 comprises a pair of batteries 730. The batteries 730 are arranged on, i.e. supported by, the protruding support portion 716 and electrically connected to the pump 705. Thus, the pump 705 is operated by electric power received from the batteries 730.

Furthermore, the container supporting portion 718 is preferably an elongated container supporting portion comprising a first end surface 731 comprising a first end 732. The elongated container supporting portion 718 also comprises a second end 734. In detail, the container supporting portion 718 is, when arranged on the user as depicted in FIG. 1A, elongated in a substantially vertical direction relative to ground between the first 732 and second 734 ends. The first end 732 also comprises a first opening 736 at which a first end 740 of the elongated tube 708 is attached. The elongated tube 708 is at a second end 742 attached to an inlet 744 of the needle insertor arrangement 100.

The second end 734 of the elongated container supporting portion 718 comprises a second opening 738. The medicament container 712, also commonly referred to as a medicament vial, is hereby insertable into the elongated container supporting portion 718 in the at least vertical direction relative to ground when the arranged on the user as depicted in FIG. 1A. A lid 746 of the medicament container 712 is preferably facing the first end 740 of the elongated container supporting portion 718 when inserting the medicament container 712 into the elongated container supporting portion 718. According to a non-limiting example embodiment, the elongated container supporting portion 718 may be at least semi-circular in a cross-section perpendicular to the elongation of the container supporting portion 718 for supporting a substantially cylindrically shaped medicament container 712.

Referring now to the user wearable garment 703. The user wearable garment 703 comprises the above described base portion 720 at which the protruding supporting portion 716 and the container supporting portion 718 are arranged. In further detail, the base portion 720 comprises a lower end portion 748 and an upper end portion 750. The lower end portion 748 is arranged vertically below the upper end portion 750 relative to ground when the user wearable garment 703 is arranged around the neck of the user 704.

The user wearable garment 703 also comprises a first 752 and a second 754 arm portion. Each of the arm portions 752, 754 are integrally formed with the base portion 720 and extends from the base portion 720 in a direction away from the lower end portion 748 of the base portion 720. Hereby, the first 752 and second 754 arm portion are, together with the upper end portion 750 of the base portion 720 are forming a closed loop portion 756 that is supportable by the neck of the user (704 in FIG. 1A).

According to the exemplified embodiment depicted in FIG. 2, the closed loop portion 756 is formed by connecting a first connector element 758 of the first arm portion 752 with a second connector element 760 of the second arm portion 754 with each other. The first connector element 758 is thus arranged on a first outer free end 762 of the first arm portion 752 while the second connector element 760 is arranged on a second outer free end 764 of the second arm portion 754. Accordingly, the first connector element 758 is arranged on the first arm portion 752 at a first position, i.e. the first outer free end 762, away from the base portion 720, and the second connector element 760 is arranged on the second arm portion 754 at a second position, i.e. the second outer free end 764, away from the base portion 720.

According to an example embodiment, and as indicated in FIG. 2, the first connector element 758 is a male connector element and the second connector element 750 is a female connector element which are arranged to mate with each other. Other alternatives are of course conceivable, such as a magnet coupling, etc.

Moreover, the infusion subassembly 702 further comprises a control unit 766 electrically connected to the pump 705. Hence, the control unit 766 is configured to control operation of the pump 705. In detail, the control unit 766 comprises control circuitry configured to transmit a control signal to the pump 705. The control signal representing instructions which, when executed by the pump 705, cause activation of the pump 705 to force transfer of medicament from the medicament container 712 out through the first opening 736 of the first end surface 731 via the elongated tube 708. The control unit 766 is preferably embedded in the user wearable garment 703, i.e. not externally accessible.

The control unit 766 may include a microprocessor, microcontroller, programmable digital signal processor or another programmable device. The control unit 766 may also, or instead, include an application specific integrated circuit, a programmable gate array or programmable array logic, a programmable logic device, or a digital signal processor. Where the control unit 766 includes a programmable device such as the microprocessor, microcontroller or programmable digital signal processor mentioned above, the processor may further include computer executable code that controls operation of the programmable device.

Also, the infusion subassembly 702 comprises an actuator 768. The actuator 768 can be arranged as a push button on the surface of the user wearable garment 703. Thus, the push button is arranged on the surface facing away from the user when the user wearable garment 703 is worn by the user. The actuator 768 is connected to the control unit 766 and the above described control signal from the control unit 766 to the pump 705 is transmitted upon actuation of the actuator 768.

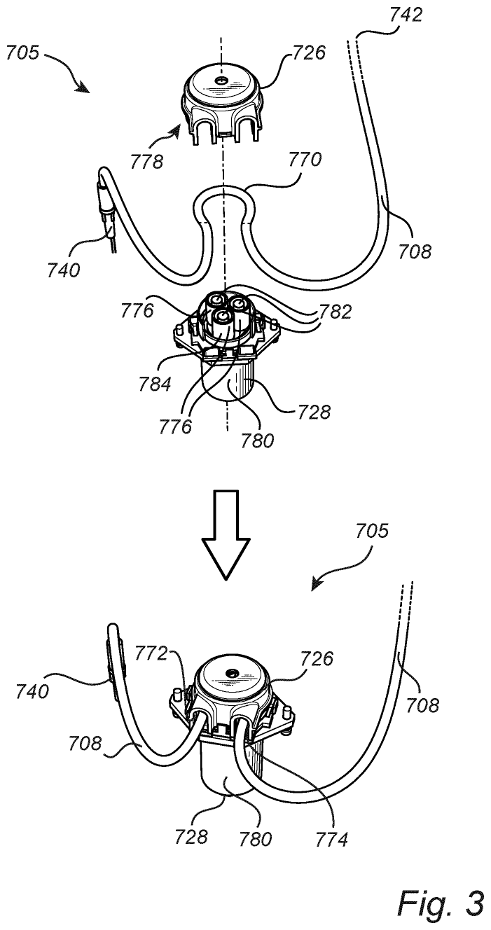

In order to describe an exemplified embodiment of the pump 705, reference is now made to FIG. 3. FIG. 3 illustrates the pump in the form of a peristaltic pump, which is depicted in an exploded view and in an assembled view according to an exemplified embodiment. In particular, FIG. 3 illustrates the pump 705 as well as the elongated tube 708. As described above, the elongated tube 608 comprises the first end 740 and a second end 742. The pump 705 comprises the above described first 726 and second 728 housing portions. A portion 770 of the elongated tube 708 is arranged in the first housing portion 726. As can be seen in FIG. 3 in combination with FIG. 2, the elongated tube 708 extends from the first opening 736 of the first end 732 of the container supporting portion 718 into the first housing portion 726. The elongated tube 708, and in particular the portion 770 of the elongated tube 708, thereafter extends within the first housing portion between a pump inlet 772 and a pump outlet 774. From the pump outlet 774, the elongated tube 708 extends to the inlet 744 of the needle insertor arrangement 100.

The pump 705 further comprises at least one engagement roller 776. The at least one engagement roller 776 is arranged in the first housing portion 726 of the pump 705. According to the exemplified embodiment depicted in FIG. 3, the pump 705 comprises three engagement rollers 776. The portion 770 of the elongated tube 708 is arranged between the engagement rollers 776 and a surface 778 of the first housing portion 726. In particular, the surface 778 of the first housing portion 726 is the surface facing the interior of the first housing portion 726.

The pump 705 also comprises an electric motor 780. The electric motor 780 is arranged in the second housing portion 728 and comprises a motor shaft (not shown) connected to the engagement rollers 776. Thus, the electric motor 780 is preferably receiving electric power from the above described batteries 730 for rotation of the engagement rollers 776. The electric motor 780 is also preferably connected to the above described control unit 766. Thus, when the actuator is actuated, the control unit 766 transmits a control signal to the electric motor 780. When receiving the control signal, the electric motor 780 is started and rotates the motor shaft and in turn the engagement rollers 776.

When the electric motor 780 is operated, each of the engagement rollers 776 rotates around a respective rotation axis 782. The portion 770 of the elongated tube 708 is hereby squeezed between the engagement roller 776 and the surface 778 of the first housing portion 726. Medicament present in the portion 770 of the elongated tube 708 is hereby forced out from the pump outlet 774 and directed towards the inlet 744 of the needle insertor arrangement 100. The medicament in the medicament container 712 will hereby also be drawn out from the medicament container 712 and forced towards the pump inlet 772. Although not depicted in FIG. 3, the portion 770 of the elongated tube 708 may as an alternative be arranged between the engagement rollers 776. During rotation of the engagement rollers 776, the portion 770 of the elongated tube 708 is instead squeezed between two engagement rollers 776. To improve the engagement between the portion 770 of the elongated tube 708 and the engagement rollers 776, the engagement rollers 776 comprises a rotatable friction surface 784. The rotatable friction surface 784 is thus configured to squeeze the portion 770 of the elongated tube 708 against the surface 778 of the first housing portion 726 upon rotation of the engagement rollers 776.

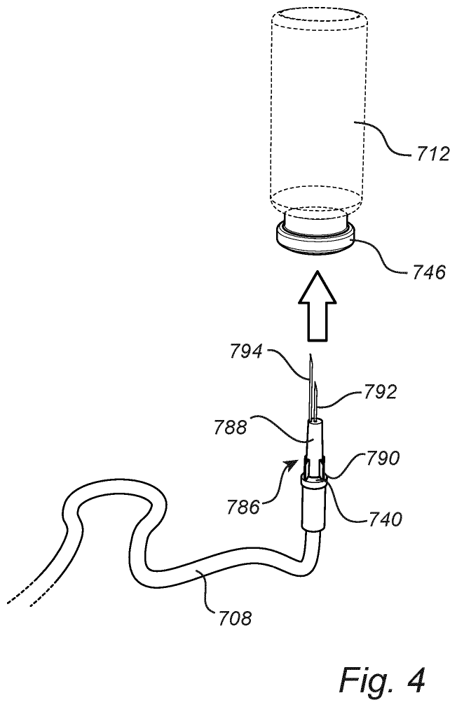

Turning now to FIG. 4 which is a schematic illustration the interaction between the medicament container 712 and the elongated tube 708 according to an exemplified embodiment. As can be seen, the first end 740 of the elongated tube 708 comprises a container connector portion 786. The container connector portion 786 is arranged to attach the first end 740 of the elongated tube 708 to the first opening 736 of the container supporting portion 718. The container connector portion 786 comprises a taper shaped surface 788 and a plurality of radially extending protrusions 790, each protrusion extends from the taper shaped surface 788. The taper shaped surface 788 and the radially extending protrusions 790 improves the attachment to the first opening 736 of the container supporting portion 718.

Furthermore, the first end 740 of the elongated tube 708 comprises a needle 792 and an aspiration needle 794. The needle 792 and the aspiration needle are configured to be directed through the first opening 738 of the container supporting portion 718 and to penetrate the lid 746 of the medicament container 712. Medicament in the medicament container 712 is configured to be forced into the elongated tube 708 through the needle 792, while the aspiration needle 794 is configured to provide air into the medicament container 712.

In order to describe the needle insertor arrangement 100 in further detail, reference is made to FIGS. 5-19. Starting with FIG. 5 which is an exploded view of a needle insertor arrangement 100 according to the first embodiment of the present disclosure. The needle insertor arrangement 100 includes a case 200, a cover 300, a plurality of fixing elements 310, an O-ring 320, a first seal 331, a second seal 332, and a third seal 333. The needle insertor arrangement 100 further includes an insertor assembly 500 positioned within the enclosure of the case 200 and cover 300. The structure of insertor assembly 500 will be further explained in detailed below.

The case 200 includes a first opening 211, a second opening 212, and a third opening 213 on the injection site abutment surface 240, wherein the injection site abutment surface 240 faces the injection site, i.e. the dose delivery site 710 when the needle insertor arrangement 100 is placed on the injection site. The first, second, and third seals 331, 332, 333 are configured to attach to the case 200 to cover the first, second, and third openings 211, 212, 213.

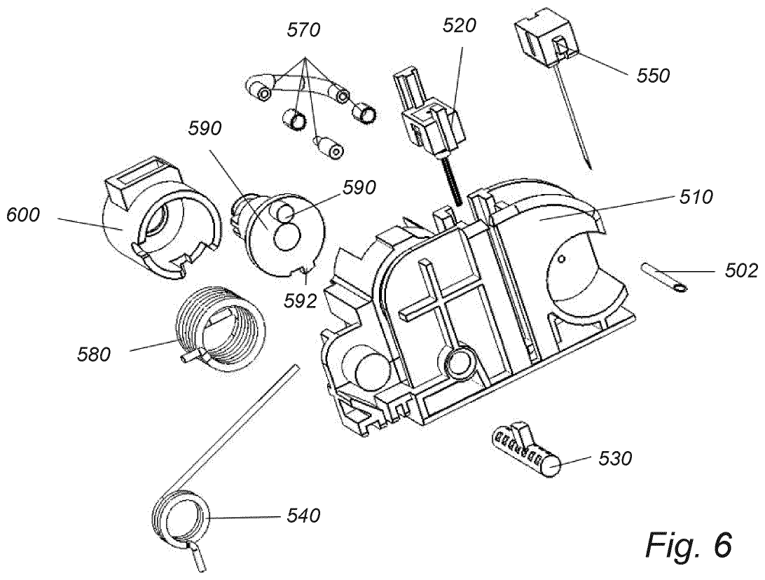

The insertor assembly 500 includes a container port 501 configured with a piercer 502 for connection to the second end 742 of the elongated tube 708. The piercer 502 is thus arranged as an inlet of the insertor assembly 500. The insertor assembly 500 further includes a movable stop 530 (illustrated in FIG. 6) configured to be moved to activate the needle insertion sequence of the insertor assembly 500. The insertor assembly 500 includes a fourth opening 214 (illustrated in FIG. 5) corresponding to the third opening 213, wherein a needle within the insertor assembly 500 will pass through the third and fourth openings 213, 214 to penetrate the injection site. When the insertor assembly 500 is positioned inside the case 200, the piercer 502 and stop 530 will be respectively exposed through the first and second openings 211, 212 of the case 200. However, the first and second seals 331, 332 cover the two openings 211, 212 to ensure that external objects will not interact with the piercer 520 and stop 530 before the needle insertor 100 is ready for use. Similarly, the third seal 333 covers the third and fourth openings 213, 214 to ensure that no external objects interact with the needle within the insertor assembly 500 before the needle insertor 100 is ready for use.

The cover 300 is configured to couple with the case 200 and covers the opening through which the insertor assembly 500 is inserted into the case 200. The cover 300 includes an O-ring portion 301 surrounded by the O-ring 320. This arrangement allows the O-ring portion 301 and O-ring 320 to create a seal with the inner surface of case 200 when inserted into the case 200. In this embodiment, the fixing elements 310 are screws configured to pass through the corresponding screws openings on the case 200 and cover 300 to secure the two components 200, 300 together. Together, case 200, cover 300, and the first, second, third seals 331, 332, 333 ensure that external objects will not make contact with insertor assembly 500 before the needle insertor 100 is ready for use. Also, other suitable fixing elements known to a skilled person such as bolts can be used to secure the case 200 and cover 300 together.

FIG. 6 shows an exploded view of the insertor assembly 500 according to the first embodiment of the present disclosure. The insert assembly 500 includes a base 510, a piercer 502, a cannula assembly 520, a movable stop 530, a driver 540, a needle assembly 550, a medicament path assembly 570, an energy accumulation member 580, a rotator 590, and a sleeve 600. The function of each component mentioned above and its relationship with the base 510 will be explained in more details below.

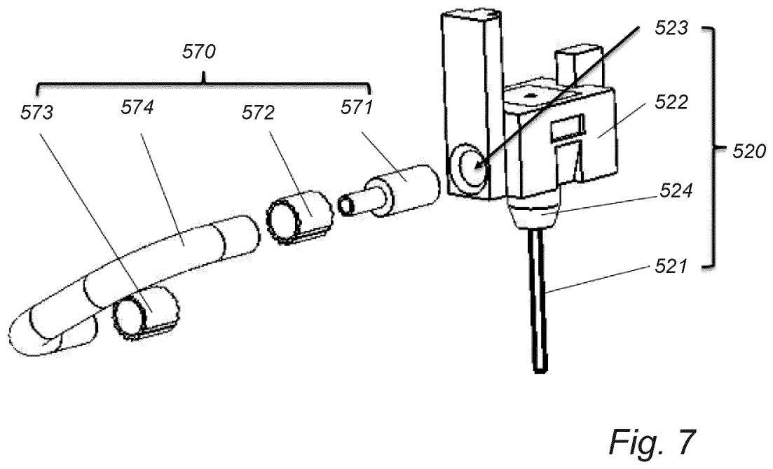

FIG. 7 shows an exploded view of the cannula assembly 520 and medicament path assembly 570. The cannula assembly 520 includes a cannula portion 521 and a cannula base portion 522. The cannula portion 521 and cannula base portion 522 are both hollow inside. The cannula base portion 522 has a hose opening 523 and a cannula mount 524 that has an opening to accommodate the cannula portion 521. The hollow cannula portion 521 is mounted on the cannula mount 524 to gain access to the inner space of the cannula base portion 522. Thus, once the cannula portion 521 is coupled with the cannula mount 524, liquid entering the hose opening 523 will be able to pass through the inner space of cannula base portion 522 and then exit through the cannula portion 521.

In the present embodiment, the medicament path assembly 570 includes an attachment pin 571, a first crimp 572, a second crimp 573, and a hose 574. The attachment pin 571 has a thicker portion configured to be fitted in the hose opening 523 to gain access to the inner space of cannula base portion 522. The attachment pin 571 also has a thinner portion configured to be coupled with one end of the hose 574, wherein the first crimp 572 surrounds the portion of hose 574 coupled with the attachment pin 571 to ensure that the two components are fastened. The second crimp 573 is configured to surround the portion of hose 574 coupled with the piercer 502 which will be explained with figure later.

In the present embodiment, the medicament path assembly 570 includes four components assembled together. In other embodiments, the medicament path assembly 570 can instead include only one tube or other number of components suitable to be assembled with the cannula assembly 520.

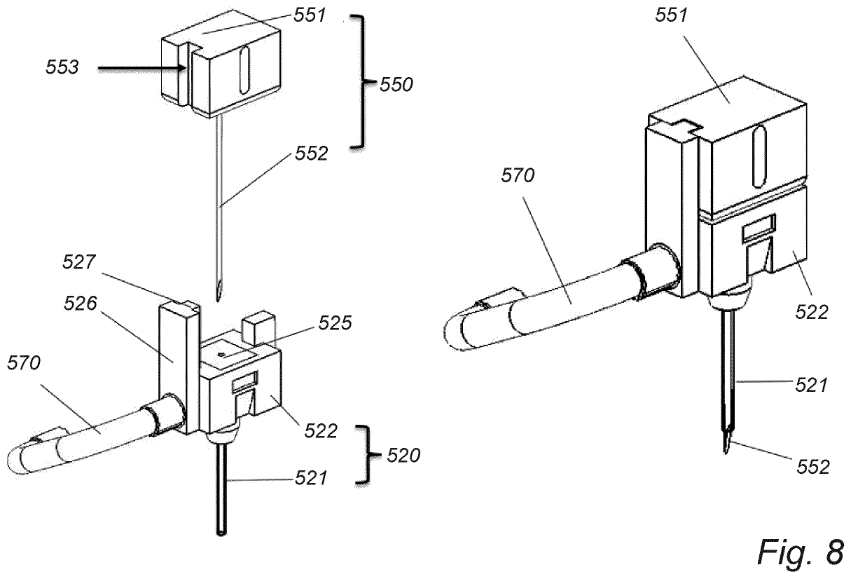

FIG. 8 shows an exploded view of the needle assembly 550, cannula assembly 520, and medicament path assembly 570 as well as a perspective view of the assembly of said components. The cannula assembly 520 and medicament path assembly 570 are assembled as described above. On the other hand, the needle assembly 550 includes a needle base portion 551 and needle portion 552 coupled together. The cannula base portion 522 includes a needle opening 525 for allowing access to the inner space of cannula base portion 522. Thus, the needle portion 552 can pass through the needle opening 525 and pass through the cannula portion 521 as illustrated on the right side of FIG. 8.

In the present embodiment, the cannula base portion 522 includes a base coupling key 526 having a protrusion 527. On the other hand, the needle base portion 551 has a coupling trough 553 configured to couple with the protrusion 527. Said structure ensures that the needle base portion 551 and cannula base portion 522 are coupled together in such as a way that external forces will not cause vibration of the needle base portion 551 that may cause damage to the needle portion 552 within the cannula portion 521. The configuration also serves to make sure that the cannula base portion 522 is coupled with a corresponding needle base portion 551. Also, in other embodiments, the protrusion 527 can be disposed on the needle base portion 551 while the corresponding coupling trough 553 is disposed on the base coupling key 526. Other suitable configuration can also be used to couple the needle base portion 551 with the cannula base portion 522.

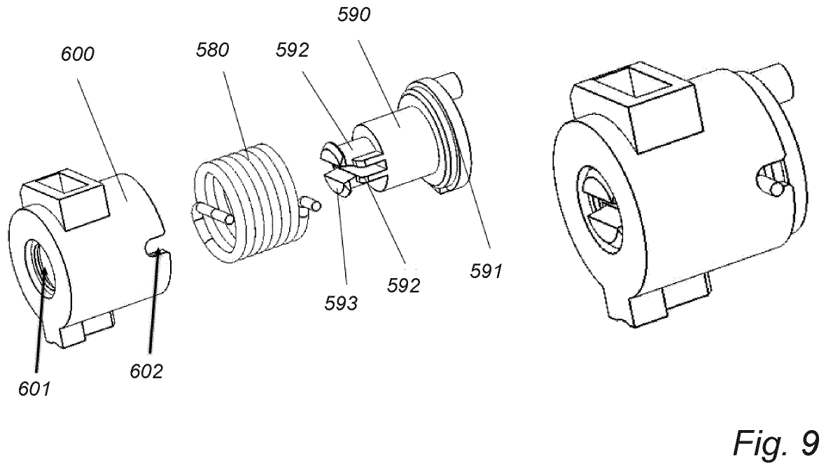

FIG. 9 shows an exploded view of the energy accumulation member 580, rotator 590, and sleeve 600 as well as a perspective view of the assembly of said components. The rotator 590 has a driver end 591 and a coupling end 592 for pass through the energy accumulation member 580 and coupling with the sleeve 600. The drive end 591 is configured to interact with the driver 540 (illustrated in FIG. 6) when the rotator 590 is released to be rotated by the energy accumulation member 580. The sleeve 600 is hollow in order to accommodate both the energy accumulation member 580 and the rotator 590. The sleeve 600 has a rotator coupling opening 601 configured to allow the coupling end 592 to pass through and couple with the sleeve 600. Each of the two coupling ends 592 has a fin 593 extending and slanting radially outward. When the coupling end 592 is pressed against the inner surface of sleeve 600, the inner surface of sleeve 600 forces the slanting surface of fin 593 and coupling end 592 as a whole to flex radially inward. In this way, both the coupling ends 592 together become thin enough to pass through the rotator coupling opening 601. Afterward, the coupling ends 592 flex radially outward and the fins 593 engage the outer surface of sleeve 600 surrounding the opening 601 to prevent the rotator 590 from being pulled away. This coupling allows the rotator 590 to be rotatable relative to the sleeve 600 and prevents the rotator 590 to move longitudinal relative to the sleeve 600. On the other hand, the energy accumulation member 580 has two ends, one positioned between the two coupling ends 592 of rotator 590 and the other positioned in a notch 602 on the sleeve 600.

In the present embodiment, the energy accumulation member 580 is a torsion spring. However, in other embodiments, the energy accumulation member 580 can be other forms of force generating component capable of accumulating energy and release the energy when released.

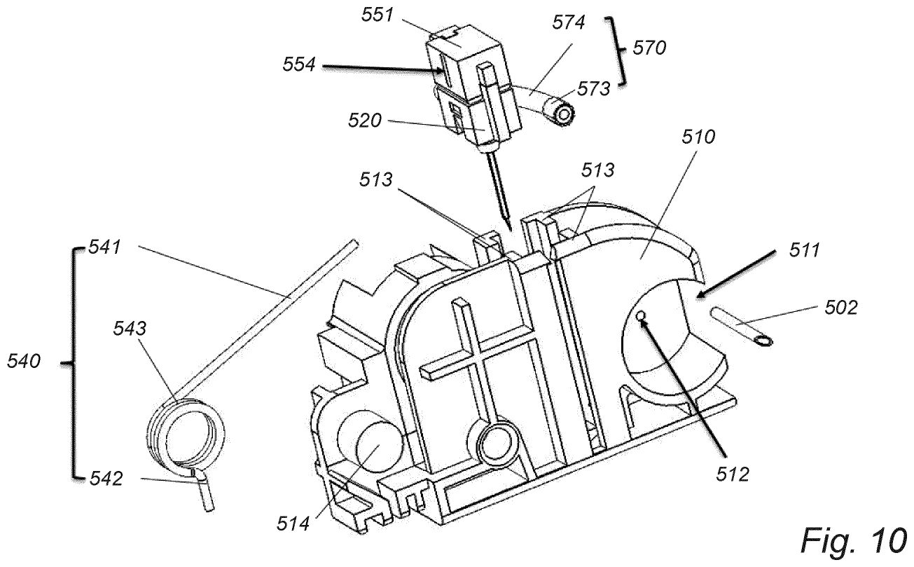

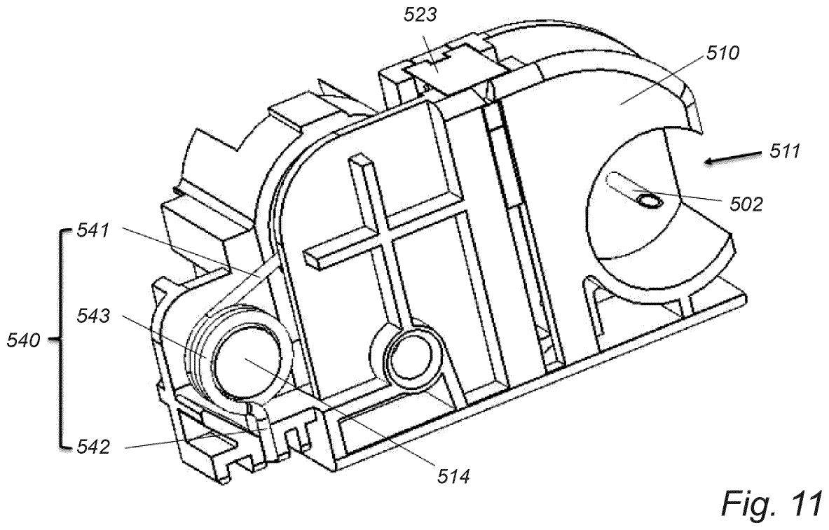

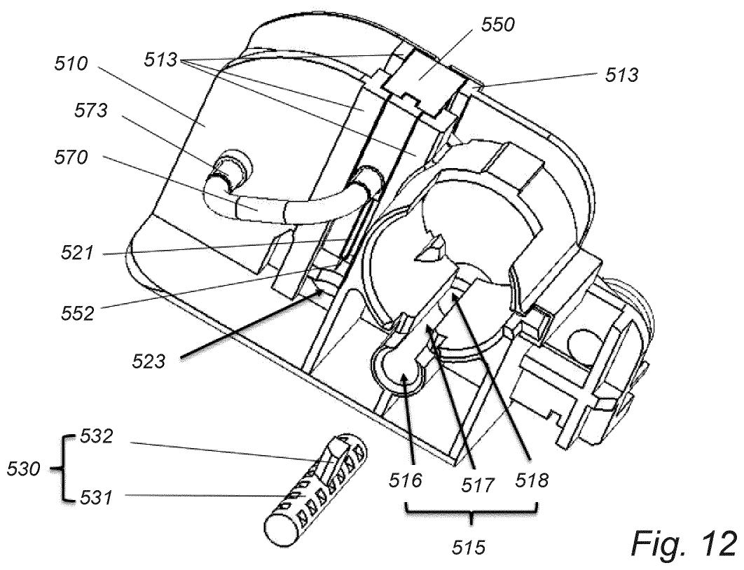

FIG. 10 shows an exploded view of the base 510, piercer 502, cannula assembly 520, driver 540, needle assembly 550, and medicament path assembly 570. FIGS. 11 and 12 show perspective views of the assembly of piercer 502, base 510, cannula assembly 520, driver 540, needle assembly 550, and medicament path assembly 570. The base 510 has a container port 511 for accommodating a medicament container and a piercer opening 512 for accommodating the piercer 502. In the present embodiment, the end of hose 574 surrounded by the second crimp 573 is positioned on one end of the piercer opening 512 while the piercer 512 passes through the other end of piercer opening 512 to be coupled with both the base 510 and hose 574. Thus, a medicament container can have its seal pierced to have the medicament within flowing through the piercer 502, hose 574, cannula base portion 522, and finally exit through the cannula portion 521.

In the present embodiment, the base 510 has a guiding structure 513 configured to accommodate and guide the insertion of the needle assembly 550 and cannula assembly 520. The guiding structure 513 creates a space with size and shape corresponding to those of the needle assembly 550 and cannula assembly 520. As illustrated in FIG. 12, the base 510 has the fourth opening 214 configured for the cannula portion 521 and needle portion 552 to pass through and reach the injection site.

As illustrated in FIGS. 10 and 11, the base 510 also has a driver rest 514 configured to couple with the driver 540. In the present embodiment, the driver 540 has a first part 541 configured to couple with the needle base portion 551, a second part 542, and a third part 543 configured to couple with the driver rest 514. As illustrated, the third part 543 of driver 540 has a circular opening with size corresponding to that of the driver rest 514. Thus, the base 510 and driver 540 can be coupled by putting the third part 543 on the driver rest 514 as illustrated in FIG. 11.

Further, as illustrated in FIG. 10, the needle base portion 551 has a driver opening 554 configured for the first part 541 of driver 540 to pass through to couple the driver 540 with the needle assembly 550. When the first part 541 is under no external forces it maintains the position of the needle assembly 550 on one end of guiding structure 513 away from the injection site opening 514, as illustrated in FIGS. 11 and 12. On the other hand, external forces may also be exerted on the first part 541 to move the needle assembly 550 toward the injection site opening 514 as will be explained later.

Here please refer to FIG. 12 for the explanation on instalment of the movable stop 530. In the present embodiment, the base 510 has a stop rest 515 configured to accommodate the movable stop 530. The stop 530 has a main portion 531 and a first obstruction portion 532. On the other hand, the stop rest 515 is tubular and has a tunnel 516 for the main portion 531 to pass through and a gap 517 for the first obstruction portion 532 to pass through. One end of the tunnel 516 is disposed with stop openings 518 so that an external object can move the stop 530 by interacting with the main portion 531 through the stop openings 518.

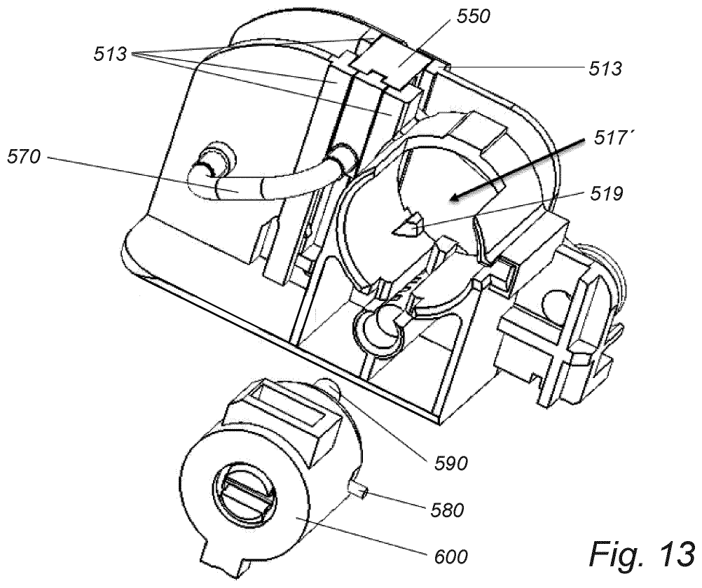

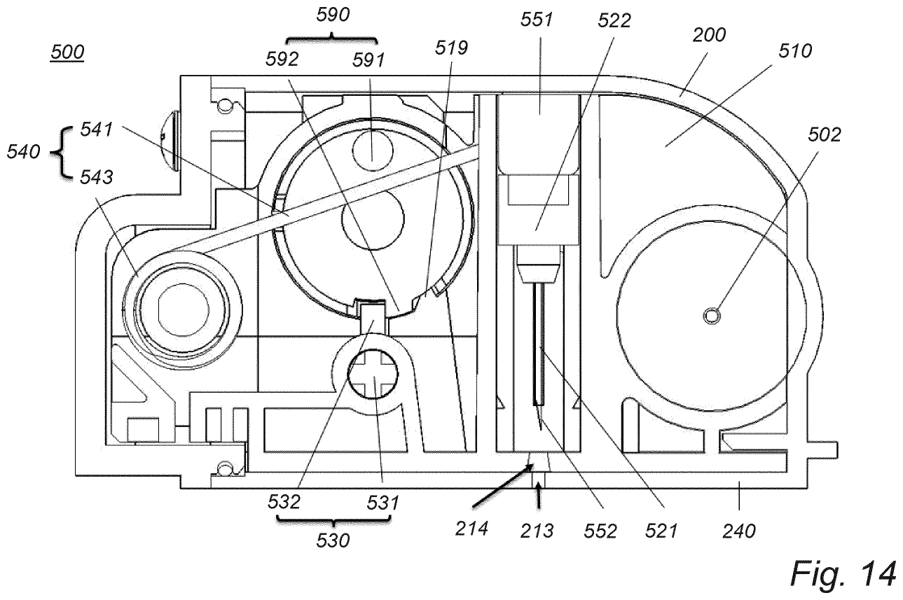

Here please refer to both FIGS. 13 and 14 for the installation of the energy accumulation member 580, rotator 590, and sleeve 600 as well as the interaction between the stop 530 and rotator 590. In the present embodiment, the base 510 has a sleeve port 517′ configured to accommodate the assembly of the energy accumulation member 580, rotator 590, and sleeve 600. The sleeve port 517′ includes a second obstruction portion 519 disposed on the inner surface of the sleeve port 517′. As illustrated in FIGS. 6 and 14, the rotator 590 includes a driving member 591 configured to interact with the first part 541 of driver 540 after the rotator 590 is released. Also as illustrated in FIG. 14, the rotator 590 also has an engagement member 592 configured to interact the first obstruction portion 532 of stop 530 before the rotator 590 is released. The engagement member 592 is also configured to interact with the second obstruction portion 519 after the rotator 590 is released. The interaction between components mentioned above will be explained in more details below.

When the assembly of the energy accumulation member 580, rotator 590, and sleeve 600 is inserted in the sleeve port 517′ of base 510, the engagement member 592 of rotator 590 will be positioned between the gap/space between the obstruction portion 518 of sleeve port 517′ and the first obstruction portion 532 of stop 530. See FIG. 14. In the present embodiment illustrated in FIG. 14, the energy accumulation member 580 constantly applies a rotational force on the rotator 590 in a clockwise direction. However, the first obstruction portion 532 prevents such clockwise rotation of the rotator 590 by engaging the engagement member 592. Unless the stop 530 is moved in order for the first obstruction portion 532 to disengage the engagement member 592, the energy accumulation member 580 will not be able to rotate the rotator 590 in a clockwise direction.

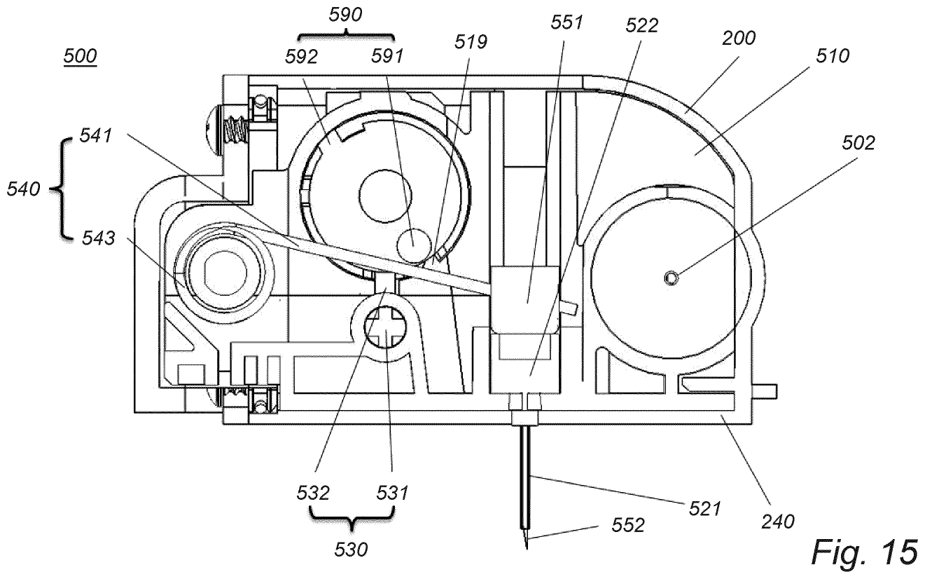

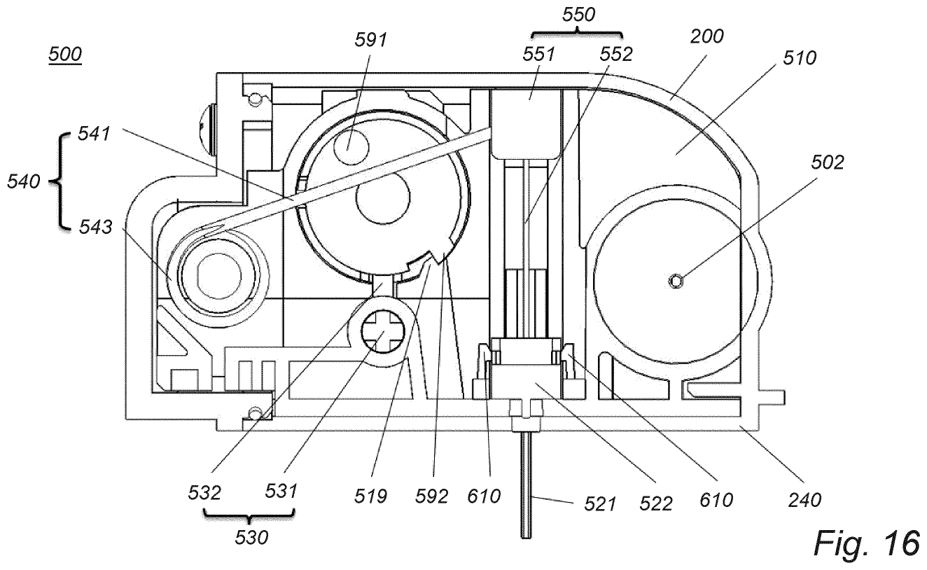

FIG. 15 shows a cross-section view of the needle insertor 100 after the stop 530 disengaged the rotator 590. An external object can be used to push the stop 530 exposed through the stop openings 518 of base 510. See FIG. 16. The result of the stop's 530 movement is that its first obstruction portion 532 is no longer in engagement with the engagement member 592 of rotator 590. Thus, the rotator 590 is promptly rotated under the rotational force of the energy accumulation member 580 in a clockwise direction. During such rotation, the driving member 591 interacts with the first part 541 of driver 540 and pushes the first part 541 downward toward the injection site abutment surface 240. As mentioned above, the first part 541 is coupled with the needle base portion 551 of needle assembly 550. Also, the needle base portion 551 is positioned above the cannula base portion 522 of cannula assembly 520. Thus, as the first part 541 is pushed downward, the first part 541 will also pushes both the cannula assembly 520 and needle assembly 550 downward. This downward movement results in both the needle portion 552 passing through the third opening 213 of case 200 and fourth opening 214 on the base 510 to pierce the injection site to create an opening. Similarly, the cannula portion 521 also passes through the injection site opening 514 and enters the injection site through the opening created by the needle portion 552. The needle penetration and cannula insertion procedures are complete.

FIG. 16 shows a cross-section view of the needle insertor 100 after the rotator 590 is prevented from further rotating by the second obstruction portion 519 of sleeve port 517. As mentioned above, the energy accumulation member 580 constantly applies rotational forces on the rotator 590, even after the needle penetration and cannula insertion procedures have been accomplished. The rotator 590 eventually rotates to the point where its engagement member 592 engages the second obstruction member 519 and the energy accumulation member 580 can no longer rotate the rotator 590. See FIG. 16. At this moment, the driving member 591 is no longer in engagement with the first part 541. Thus, the resilient nature of the driver 540 allows the first part 541 to move upward. Since the first part 541 is coupled with the needle base portion 551, this movement also brings the needle assembly 550 as a whole upward.

On the other hand, the base 510 has a pair of locking members 610 disposed on its inner surface next to the injection site opening 514. In the present embodiment, the locking members 610 has the shape of a hook. When the cannula assembly 520 is pushed toward the injection site opening 514, its cannula base portion 522 will push the locking members 610 radially outward in order to reach the inner surface of base 510. As the cannula base portion 522 reaches the inner surface of base 510, the locking members flex radially inward and then engage the cut-outs on the outer surface of cannula base portion 522. The hook shape of locking members 610 ensures that the cannula assembly 520 will not be brought back up together with the needle base portion 551 by the first part 541. Accordingly, the cannula assembly 520 and needle assembly 550 will separate when the first part 541 returns to its initial position. The needle retraction and cannula locking procedures are complete.

Now that the needle retraction and cannula locking procedures are complete, the needle portion 552 no longer occupies the space in the cannula portion 521. Thus, after a medicament container is inserted in the container port 501 to have its seal pierced by the piercer 502, the medicament within can flow through the piercer 502, hose 574, cannula base portion 522, cannula portion 521, and eventually enter the injection site.

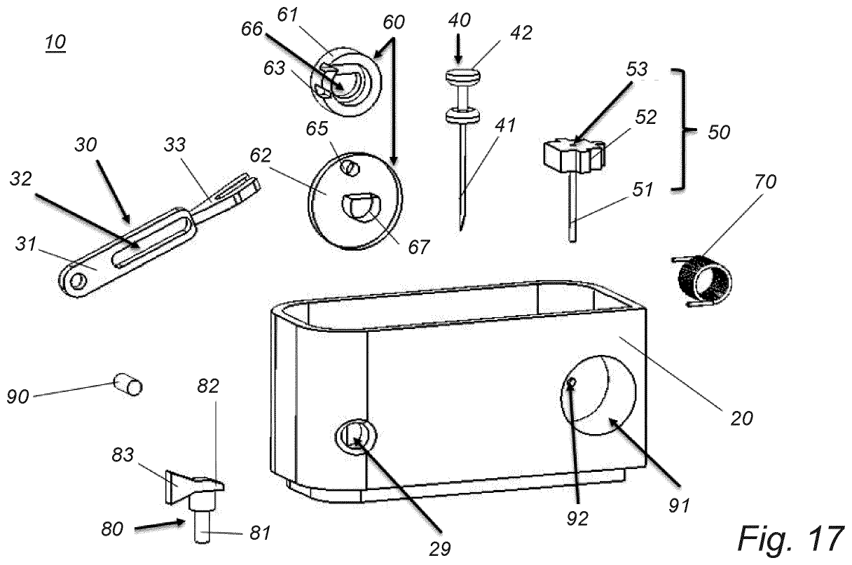

FIG. 17 shows an exploded view of an example needle insertor arrangement 10 according to a second embodiment of the present disclosure. The needle insertor arrangement 10 includes a case 20, a driver arm 30, a needle assembly 40, a cannula assembly 50, a rotator 60, a torsion spring 70, a stop member 80, and a connection member 90, wherein the rotator 60 includes a first rotator 61 and a second rotator 62. The second rotator 62 is made transparent to facilitate illustration.

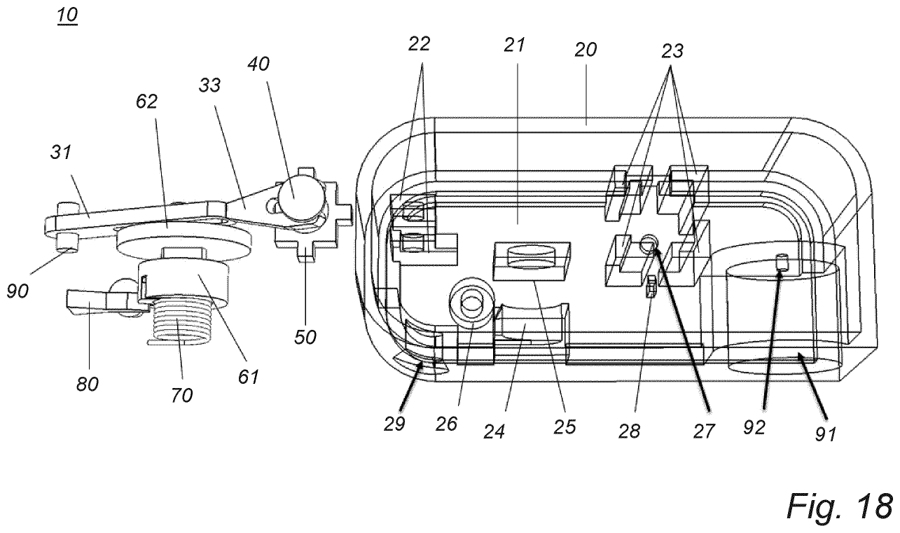

On the other hand, FIG. 18 shows a perspective view of the needle insertor 10 according to the second embodiment of the present disclosure. The case 20 is made transparent to facilitate the illustration. The case 20 includes a base 21 to accommodate the rest of the needle insertor 10, a driver arm portion 22 for coupling with one end of the driver arm 30, a guide structure 23 for accommodating the needle assembly 40 and the cannula assembly 50 and guiding the movement thereof, a first rotator stand 24 for accommodating the first rotator 61, a second rotator stand 25 for accommodating the second rotator 62, and a stop member stand 26 for accommodating the stop member 80. The base 21 includes a needle opening 27 for the needle assembly 40 and cannula assembly 50 to pass through and exits outside the case 20. The base 21 also includes a locking member 28 for later fixing the cannula assembly 50 on the base 21. The case 20 also includes an activation member opening 29 configured for an activation member (not illustrated) to pass through and interact with the stop member 80 and then release the rotator 60. The interaction between the elements on the case 20 with the rest of the needle insertor 10 will be further explained later.

In the present embodiment, the torsion spring 70 is accommodated in the space (illustrated in FIG. 17) carved out of the first rotator 61 and constantly exerts a rotational force on the first rotator 61. The first rotator 61 and torsion spring 70 are both placed on the first rotator stand 24.

As illustrated in FIG. 17, the stop member 80 has a first protrusion 81 configured to be inserted into the opening of the stop member stand 26 (illustrated in FIG. 18) in order to couple the stop member 80 with the base 21. However, the first protrusion 81 is not fixed within the stop member stand 26 and the stop member 80 is rotatable with respect to the axis of the stop member stand 26. The stop member 80 has a second protrusion 82 configured to couple with the first rotator 61 in order to prevent the rotator 60 as a whole from being rotated by the torsion spring 70. As illustrated in FIG. 17, the first rotator 61 includes a stop groove 63 configured to accommodate the second protrusion 82. As described above, the torsion spring 70 constantly applies rotational force on the first rotator 60. The second protrusion 82 can absorb the rotational force and prevent the first rotator 60 from rotating. The stop member 80 also has a third protrusion 83 configured to be pushed in order to rotate the second protrusion 82 in either a clockwise or anticlockwise direction. The purpose of the third protrusion 83 is to be rotated by user to subsequently rotate the second protrusion 82 out of the stop groove 63 in order to release the first rotator 61. As described above, the case 20 also includes an activation member opening 29 configured for an activation member (not illustrated) to pass through. The activation member can push the third protrusion 83 to rotate the second protrusion 82 out of the stop groove 63 so that the rotator 60 can be released and then rotated by the torsion spring 70.

The case 20 includes a container port 91 with a piercer opening 92 can be fitted with a piercer similar to the piercer 502 in the first embodiment. A medicament container can be inserted in the container port 91 to have its seal pierced so that the medicament within can flow through the piercer to reach the interior of case.

The first rotator 61 and second rotator 62 are meant to be coupled together so that the rotational force of the torsion spring 70 can rotate both rotators 61, 62 simultaneously. The first rotator 61 has a rotator opening 66 and the second rotator 62 has a corresponding rotator protrusion 67 configured to be fitted into the rotator opening 66. The shape of both the rotator opening 66 and rotator protrusion 67 are not circular so that the rotational force from the torsion spring 70 can be transferred from the first rotator 61 to the rotator protrusion 64 and then the second rotator 62 as a whole.

In the present embodiment, one first portion 31 of the driver arm 30 is rotatably coupled with the driver arm portion 22 of the case 20. The first portion 31 has an opening configured to be aligned with the two openings of the driver arm portion 22. The connection member 90 is then fitted in the space of the three openings in order to couple the driver arm 30 with the driver arm portion 22. Also, the connection member 90, opening of the first portion 31, and opening of the corresponding driver arm portion 22 preferably have circular shapes or other suitable shape in order for the first portion 31 to be rotatably coupled with the driver arm portion 22.

The driver arm 30 further includes a cam opening 32 and the second rotator 62 includes a cam 65 configured to be fitted within the cam opening 32. As described above, the rotational force from the torsion spring 70 rotates both the first rotator 61 and second rotator 62. The cam 65 fitted inside the opening 32 allows the second rotator 62 to directly interact with the driver arm 30. In this way, the rotational force can be transferred from the cam 65 to the driver arm 30. However, since the first portion 31 is coupled with the base 21 of the case 20, the rotational force from the cam 65 allows the cam 65 to travel within the cam opening 32 and will only pivot the driver arm 30 upward and downward. Thus, as long as the torsion spring 70 keeps the rotator 60 rotating, the driver arm 30 will continuously be pivoted upward and downward.

The introducer needle 40 includes a needle portion 41 and a needle base portion 42 located at one end of the needle portion 41. On the other hand, the driver arm 30 has a second portion 33 configured to grip the base portion 42. As the driver arm 30 is pivoted downward by the rotational force from the rotator 60, the second portion 33 will drive the base portion 42 as well as needle assembly 40 as a whole downward toward the base 20 so that the needle portion 41 can pass through the needle opening 27 on the base 21 to initiate needle penetration.

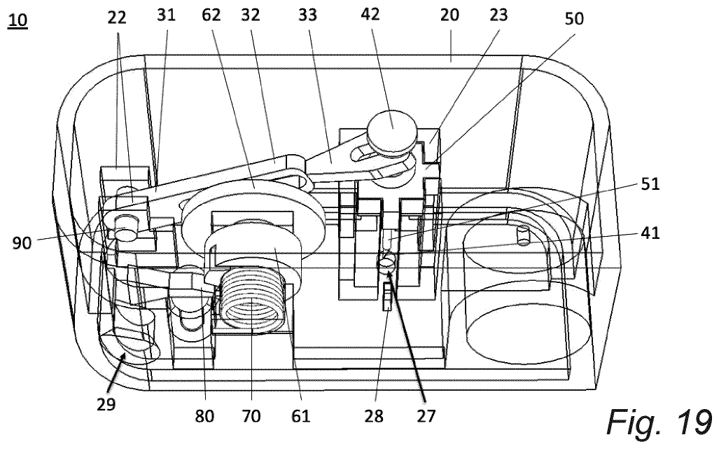

The cannula 50 includes a cannula portion 51 and a cannula base portion 52 located at one end of the cannula portion 51. The cannula base portion 52 has a first opening 53 that allows the needle portion 41 of the introducer needle 40 to pass through. The cannula portion 51 is hollow inside and the space inside the cannula portion 51 is connected to the first opening 53 of the cannula base portion 52. Also, the needle portion 41 is longer than the cannula portion 51. Thus, the needle portion 41 of the needle assembly 40 can go all the way through the cannula portion 51 so that its sharp end can emerge outside the cannula portion 51. See FIG. 19. As the needle portion 41 pass through the cannula portion 51, the base portion 42 of the needle assembly 40 will abut the cannula base portion 52 of the cannula 50. Afterwards, the force moving the needle assembly 40 will be transferred to the cannula assembly 50 and move the needle assembly 40 and cannula assembly 50 together downward toward the base 21. Also, as illustrated in FIG. 18, the guide structure 23 is configured to accommodate the shape of the base portion 52 of the cannula 50.

The guide structure 23 has gaps corresponding to the three protrusions on the cannula base portion 52. Thus, each of the protrusions is only allowed to move with the corresponding gap. The shape of the central passage of the guide structure 23 also corresponds in shape to that of the cannula base portion 52. The reason for the guide structure 23 to have shapes corresponding to those of the base portion 52 is to ensure that the needle assembly 40 and cannula assembly 50 remain at least substantially perpendicular to the base 21 at all times. Also, the cannula base portion 52 includes a second opening (not illustrated) configured to be connected with a medicament tube for medicament to pass through. The second opening is connected to the cannula portion 51. Thus, the medicament passing through the second opening can enter the cannula base portion 52, the cannula portion 51, and eventually the injector site.

As mentioned above, the container port 91 with a piercer allows a medicament container to be inserted to have its seal pierced so that the medicament within can flow through the piercer to reach the inside of case. A tube can be used to connect with the piercer and the second opening of cannula base portion 52. In this way, medicament within the container can flow through the piercer, tube, cannula base portion 52, and cannula portion 51 to eventually enter the injection site.

Initially, the second protrusion 82 of the stop member 80 is located in the stop groove 63 of the first rotator 61 to absorbs the torsion spring's forces and prevent the rotator 60 from rotating. Until the second protrusion 82 moves out of the stop groove 63, nothing will happen.

Then, an activation member pass through the activation opening 29 of the case 20 to push the third protrusion 83 and rotate the second protrusion 82 out of the stop groove 63. The moment the second protrusion 82 leaves the stop groove 63, the forces of the torsion spring 70 will force the first rotator 61 and the second rotator 62 to start rotating. As the second rotator 62 rotates, cam 65 of the second rotator 62 located inside the cam opening 32 will transfer the forces to the driver arm 30 to press the driver arm 30 downward toward the base 21. Also, since the first portion 31 of the driver arm 30 is fixed on the driver arm portion 22 of the case 20, the driver arm 30 as a whole will be pivoted relative to the driver arm portion 22. The forces on the second portion 33 are transferred to base portion 42 of the needle assembly 40 and subsequently the cannula base portion 52 of the cannula assembly 50. The forces move both the needle assembly 40 and cannula 50 downward toward the base 21 and the two components are maintained perpendicular to the base 20 due the guidance of the guide structure 23 surrounding the two components.

The needle portion 41 of the needle assembly 40 will pass through the needle opening 27 on the base 21 to create an opening by piercing an injector site which is typically the skin of the medicament recipient (706 in FIGS. 1A and 1B). The cannula portion 51 then follows the needle portion 41 and enters the injection site. Further, as the cannula base portion 52 of the cannula 50 reaches its lower position and substantially makes contact with the base 21. The locking member 28 on the base 21 will couple with the cannula base portion 52 to fix the cannula base portion 52 on the base 21 and the cannula portion 51 at least partly within the injector site.

The locking member 28 of the present embodiment is similar to the locking member 610 in the previous embodiment and has the shape of a hook. When the cannula assembly 50 is pushed toward the base 21, its cannula base portion 52 will push the locking members 28 outward in order to reach the base 21. As the cannula base portion 52 reaches the base 21, the locking members 28 returns to their initial positions and respectively engages the cannula base portion 52. The hook shape of locking members 28 ensures that the cannula assembly 50 will not be brought back up later with the needle assembly 40 by the driver arm 30. In other words, the cannula assembly 50 and needle assembly 40 will separate when driver arm 30 returns to its initial position.

After the needle portion 41 passes through the needle opening 27 on the base 21, the torsion spring 70 continues to rotate the rotator 60 and the cam 65 of the second rotator 62 can continue to pivot the driver arm 30. Afterward, the forces from the torsion spring 70 will force the cam 65 within the cam opening 32 to pivot the driver arm 30 upward and away from the base 21. Since the second portion 33 of the driver arm 30 is coupled with the base portion 42 of the needle assembly 40, the driver arm 30 will retract the needle assembly 40 back into the case 20.

During this movement, the needle portion 41 is pulled out of the injection site and passes through the space within the cannula 50 to create a clear passage from the second opening of the base portion 52 to the opening of the cannula portion 51. In this way, the medicament can then flow through the second opening 55 and exit the cannula portion 51 in order to enter the injection site for medicament injection. At this stage, the needle insertor 10 has accomplished its purpose of creating an opening on the injection site using the needle assembly 40, inserting the cannula 50 into the injection site, and retracting the needle assembly 40 back into the case 20 in order to create a passage for medicament to flow through the cannula 50 and enter the injection site.

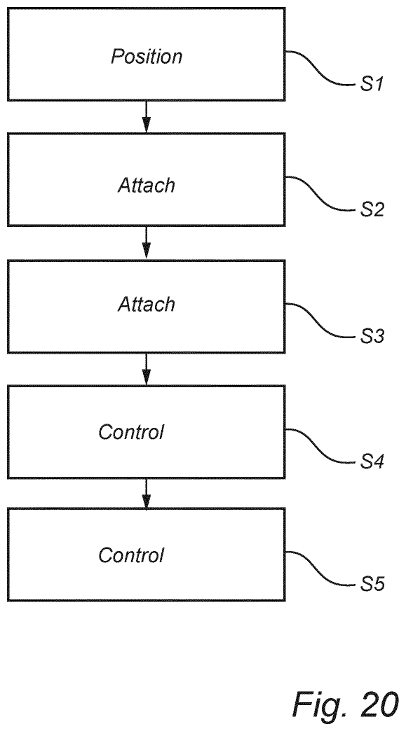

Reference is now finally made to FIG. 20. FIG. 20 is a flow chart of a method of controlling medicament delivery to a medicament recipient according to an exemplified embodiment. When operating the above described infusion assembly 700, the injection site abutment surface 240 of the needle insertor arrangement 100 is positioned S1 in abutment with a skin surface of a medicament recipient subject 706 to medicament treatment. The first end 740 of the elongated tube 708 is attached S2 to the medicament container 712 and the second end 742 of the elongated tube 708 is attached S3 to the inlet 744 of the needle insertor arrangement 100.

Thereafter, the needle insertor arrangement 100 is controlled S4 to force a penetration needle, i.e. the above described needle portion 552, of the needle insertor arrangement 100 to protrude from the injection site abutment surface 240. The pump 705 is thereafter controlled S5 to transfer medicament from the medicament container 712 to the penetration needle via the inlet portion of the needle insertor arrangement.

The inventive concept has mainly been described above with reference to a few examples. However, as is readily appreciated by a person skilled in the art, other embodiments than the ones disclosed above are equally possible within the scope of the inventive concept, as defined by the appended claims.

Some aspects of the invention are described in the clauses below.

1. An infusion subassembly, comprising:
a pump, and
a user wearable garment, comprising a protruding support portion supporting the pump to the user wearable garment, and a container supporting portion, the container supporting portion being configured to support a medicament container to the user wearable garment, wherein the container supporting portion comprises an end surface, the end surface comprising an end portion and an opening,
wherein the pump is connectable to the end portion of the end surface, and wherein the pump is configured to force medicament from the medicament container to be dispensed through the opening of the end surface.

2. The infusion subassembly according to clause 1, wherein the pump is connectable to the end portion of the container supporting portion by an elongated tube between the pump and the end portion.

3. The infusion subassembly according to any one of clauses 1 or 2, wherein the protruding support portion and the container supporting portion are integrally formed with the user wearable garment.

4. The infusion subassembly according to any one of the preceding clauses, further comprising a control unit connected to the pump, wherein the control unit comprises control circuitry configured to transmit a control signal to the pump, the control signal representing instructions which, when executed by the pump, cause activation of the pump to force transfer of medicament from the medicament container out through the opening of the end surface.

5. The infusion subassembly according to clause 4, wherein the control unit is embedded in the user wearable garment.

6. The infusion subassembly according to any one of clauses 4 or 5, further comprising an actuator arranged on the user wearable garment, the actuator being connected to the control unit, and wherein the control signal to the pump is transmitted upon actuation of the actuator.

7. The infusion subassembly according to any one of the preceding clauses, further comprising at least one battery connectable to the pump, wherein the battery is supported by the protruding support portion.

8. The infusion subassembly according to any one of the preceding clauses, wherein the pump comprises a pump inlet connectable to the end portion of the container supporting portion.

9. The infusion subassembly according to clause 8 when dependent on clause 2 or according to clause 8 when dependent on any one of clause 3-7 that is dependent on clause 2, wherein the pump comprises at least one engagement roller, the at least one engagement roller being configured to, during rotation around a rotation axis of the at least one engagement roller, squeeze a portion of the elongated tube.

10. The infusion subassembly according to clause 9, wherein the pump comprises a first housing portion configured to house the portion of the elongated tube, wherein the at least one engagement roller is configured to squeeze the portion of the elongated tube against a surface of the first housing portion.

11. The infusion subassembly according to clause 9, wherein the at least one engagement roller is formed by a first engagement roller and a second engagement roller, the first and second engagement rollers being configured to, during rotation of the first engagement roller around a first rotation axis and rotation of the second engagement roller around a second rotation axis, squeeze the portion of the elongated tube between the first and second engagement rollers.

12. The infusion subassembly according to any one of clauses 9-11, wherein the pump further comprises an electric motor connected to the at least one engagement roller.

13. The infusion subassembly according to clause 12, wherein the electric motor comprises a motor shaft connected to the at least one engagement roller for rotating the at least one engagement roller.

14. The infusion subassembly according to any one of clauses 12 or 13, wherein the pump comprises a second housing portion, the electric motor being arranged in the second housing portion.

15. The infusion subassembly according to clause 14, wherein the protruding support portion comprises a pump supporting surface, wherein the pump is arranged in an opening of the pump supporting surface, and wherein the first and second housing portions are arranged on opposite sides of the pump supporting surface.

16. The infusion subassembly according to any one of clauses 9-15, wherein the at least one engagement roller comprises a rotatable friction surface, the rotatable friction surface being configured to squeeze the portion of the elongated tube against the surface of the first housing portion upon rotation of the at least one engagement roller.

17. The infusion subassembly according to any one of clauses 9-16, wherein the pump is a peristaltic pump.

18. The infusion subassembly according to any one of the preceding clauses, wherein the container supporting portion is an elongated container supporting portion, and wherein the opening is a first opening, and the end portion is a first end, the elongated container supporting portion further comprising a second opening at a second end, the elongated container supporting portion extending between the first end and the second end, wherein the medicament container is insertable to the elongated container supporting portion from the second opening.

19. The infusion subassembly according to clause 18, wherein the elongated container supporting portion extending, during use of the infusion subassembly, between the first end and the second end in an at least partially vertical direction relative to ground.

20. The infusion subassembly according to any one of the preceding clauses, wherein the user wearable garment comprises a base portion, and two arm portions, wherein each arm portion extends from the base portion.

21. The infusion subassembly according to clause 20, wherein the base portion and the arm portions are integrally formed with each other.

22. The infusion subassembly according to any one of clauses 20 or 21, wherein the protruding support portion and the container supporting portion are arranged on the base portion.

23. The infusion subassembly according to clause 22, wherein the protruding support portion and the container supporting portion are arranged on a lower end portion of the base portion and the two arm portions are arranged on an upper end portion of the base portion, the upper and lower end portions being arranged on opposite vertical ends of the base portion, wherein the two arms extend in a direction away from the lower end portion.

24. The infusion subassembly according to any one of clauses 20-23, wherein the two arm portions are, together with the base portion, configured to form a closed loop.

25. The infusion subassembly according to clause 24, wherein a first one of the two arm portions comprises a first connector element, and wherein a second one of the two arm portions comprises a second connector element, the first and second connector elements being connectable to each other to form the closed loop.

26. The infusion subassembly according to clause 25, wherein the first connector element is arranged on one of the two arm portions at a first position away from the base portion, and wherein the second connector element is arranged on the other once of the two arm portions at a second position away from the base portion.

27. The infusion subassembly according to clause 26, wherein the first connector element is arranged at an outer end portion of the one of the two arm portions, and the second connector element is arranged at an outer end portion of the other one of the two arms portion.

28. The infusion subassembly according to any one of clauses 25-27, wherein the first connector element is a male connector element and the second connector element is a female connector element, the male connector element is arranged to mate with the female connector element.

29. The infusion subassembly according to clause 1, wherein the pump is directly connectable to the end portion of the container supporting portion.

30. The infusion subassembly according to clause 29, wherein the pump is an air compressor pump, the end surface of the container supporting portion comprising an additional opening arranged adjacent the opening, wherein an air outlet portion of the air compressor is connectable to the additional opening for supplying pressurized air to the medicament container.

31. An infusion assembly, comprising
an infusion subassembly according to any one of the preceding clauses,
a medicament container supported by the container supporting portion and connected to the pump,
a needle insertor arrangement comprising an inlet, and
an elongated tube extending between a first end and a second end, wherein the first end is attached to the end portion of the container supporting portion, and the second end is attached to the inlet of the needle insertor arrangement.

32. The infusion assembly according to clause 31, wherein an aspiration needle is attached to the first end of the elongated tube, the aspiration needle being penetrated through a lid of the medicament container.

33. The infusion assembly according to any one of clauses 31 or 32, wherein the needle insertor arrangement comprises an injection site abutment surface configured to be arranged in abutment with a medicament recipient subject to medicament treatment.

34. A method of controlling medicament delivery to a medicament recipient using an infusion assembly according to any one of clauses 31-33,
wherein the method comprises the steps of:
   positioning (S1) an injection site abutment surface of the needle insertor arrangement in abutment with a skin surface of a medicament recipient subject to medicament treatment,
   attaching (S2) the first end portion of the elongated tube to the medicament container,
   attaching (S3) the second end portion of the elongated tube to the inlet portion of the needle insertor arrangement;
   controlling (S4) the needle insertor arrangement to force a penetration needle of the needle insertor arrangement to protrude from the injection site abutment surface; and
   controlling (S5) the pump to transfer medicament from the medicament container to the penetration needle via the inlet portion of the needle insertor arrangement.

35. The method according to clause 34, wherein the step of attaching the second end portion of the elongated tube to the inlet portion of the needle insertor arrangement is preceded by the steps of:
verifying a flow of medicament from the medicament container through the elongated tube by activating the pump; and
de-activating the pump when medicament flows out from the second end portion of the elongated tube.

1-15. (canceled)

16. An infusion subassembly, comprising:
a pump; and
a user wearable garment, including a protruding support portion supporting the pump to the user wearable garment and a container supporting portion, the container supporting portion being configured to support a medicament container to the user wearable garment, wherein the container supporting portion comprises an end surface, the end surface comprising an end portion and an opening,
wherein the pump is connectable to the end portion of the end surface, and wherein the pump is configured to force medicament from the medicament container to be dispensed through the opening of the end surface.

17. The infusion subassembly according to claim 16, wherein the pump is connectable to the end portion of the container supporting portion by an elongated tube between the pump and the end portion.

18. The infusion subassembly according to claim 16, wherein the protruding support portion and the container supporting portion are integrally formed with the user wearable garment.

19. The infusion subassembly according to claim 16, further comprising a control unit connected to the pump, wherein the control unit comprises control circuitry configured to transmit a control signal to the pump, the control signal representing instructions which, when executed by the pump, cause activation of the pump to force transfer of medicament from the medicament container out through the opening of the end surface.

20. The infusion subassembly according to claim 19, wherein the control unit is embedded in the user wearable garment.

21. The infusion subassembly according to claim 16, wherein the pump is a peristaltic pump.

22. The infusion subassembly according to claim 16, wherein the container supporting portion is an elongated container supporting portion, and wherein the opening is a first opening, and the end portion is a first end, the elongated container supporting portion further comprising a second opening at a second end, the elongated container supporting portion extending between the first end and the second end, wherein the medicament container is insertable to the elongated container supporting portion from the second opening.

23. The infusion subassembly according to claim 22, wherein the elongated container supporting portion extending, during use of the infusion subassembly, between the first end and the second end in an at least partially vertical direction relative to ground.

24. The infusion subassembly according to claim 16, wherein the user wearable garment comprises a base portion, and two arm portions, wherein each arm portion extends from the base portion.

25. The infusion subassembly according to claim 24, wherein the base portion and the arm portions are integrally formed with each other.

26. The infusion subassembly according to claim 24, wherein the two arm portions are, together with the base portion, configured to form a closed loop.

27. An infusion assembly, comprising
the infusion subassembly according to claim 16,
a medicament container supported by the container supporting portion and connected to the pump,
a needle insertor arrangement comprising an inlet, and
an elongated tube extending between a first end and a second end, wherein the first end is attached to the end portion of the container supporting portion, and the second end is attached to the inlet of the needle insertor arrangement.

28. The infusion assembly according to claim 27, wherein an aspiration needle is attached to the first end of the elongated tube, the aspiration needle being penetrated through a lid of the medicament container.

29. The infusion assembly according to claim 27, wherein the needle insertor arrangement comprises an injection site abutment surface configured to be arranged in abutment with a medicament recipient subject to medicament treatment.

30. A method of controlling medicament delivery to a medicament recipient using an infusion assembly according to claim 27,
wherein the method comprises the steps of:
positioning an injection site abutment surface of the needle insertor arrangement in abutment with a skin surface of a medicament recipient subject to medicament treatment,
attaching the first end portion of the elongated tube to the medicament container,
attaching the second end portion of the elongated tube to the inlet portion of the needle insertor arrangement;
controlling the needle insertor arrangement to force a penetration needle of the needle insertor arrangement to protrude from the injection site abutment surface; and
controlling the pump to transfer medicament from the medicament container to the penetration needle via the inlet portion of the needle insertor arrangement.
